(19) United States
(12) Patent Application Publication
(10) Pub. No.: US 2019/0290388 A1
(43) Pub. Date: Sep. 26, 2019

(54) AIR PLENUMS FOR PRODUCING IMPROVED LAMINAR ENTRAINMENT AIRFLOW AND METHODS OF THEIR USE

(71) Applicant: Optimus Licensing AG, Zug (CH)

(72) Inventor: John R. Mangiardi, Zug (CH)

(21) Appl. No.: 16/360,754

(22) Filed: Mar. 21, 2019

Related U.S. Application Data
(60) Provisional application No. 62/646,272, filed on Mar. 21, 2018.

(51) Int. Cl. A61B 90/40; A61G 13/10

(52) U.S. Cl. CPC A61B 90/40; A61B 2090/401; A61G 13/108

(57) ABSTRACT

Air plenums that produce highly laminar airflow and methods of their use are disclosed. In certain embodiments, an air plenum produces substantially downward laminar airflow such that a vertical curtain of air extending down from the plenum is created (e.g., at least in part by entrainment). In certain embodiments, an average airflow velocity in an outer region of airflow is at least 1.5× higher than an average airflow velocity of an inner region surrounded by the outer region, for example, when measured at a certain height above the floor (e.g., at a height of a patient treatment table). In certain embodiments, outlets of an air plenum are arranged such that they are bounded by a circular cross section. As a circular cross section does not have any corners, turbulent regions at the perimeter of the downward airflow are eliminated.

### PRIORITY APPLICATION

The present application claims the benefit of U.S. Provisional Patent Application No. 62/646,272, filed Mar. 21, 2018, entitled Air Plenums for Producing Improved Laminar Airflow and Methods of Their Use, the disclosure of which is hereby incorporated by reference herein in its entirety.

### CROSS REFERENCE TO RELATED APPLICATIONS

This application is related to U.S. Provisional Patent Application No. 62/427,773, filed Nov. 29, 2016, entitled Systems and Apparatus for Reducing Risks in Integrated Healthcare Settings and Methods of Their Use, and PCT Application No. PCT/EP2017/078636, filed Nov. 8, 2017, entitled Integrated Operating Room Lighting and Patient Warming System—Design and Components, each of which is hereby incorporated by reference herein in its entirety.

### TECHNICAL FIELD

The present disclosure relates generally to air plenums for disposal in ceilings (e.g., of healthcare settings), wherein the air plenums produce downward laminar airflow.

### BACKGROUND

The cleanliness and sterility of healthcare settings is important for patient health. Maintaining clean and, in some cases, sterile healthcare settings (e.g., a sterile operating room) reduces risks that patients become infected during or as a result of treatment in a healthcare setting. For example, doctors and nurses engage in frequent hand washing to reduce transmission of infectious diseases and instruments (e.g., surgical tools) used in patient treatment are sterilized prior to use. Nevertheless, infectious diseases still exist in healthcare environments such as hospitals and clinics. Airflow is commonly used to prevent transmission of such infectious disease to a patient, who may have a compromised ability to fight any infection. For example, in a typical operating room, airflow from the ceiling is directed downward at a moderate to high rate in order to push any airborne microbes away from a patient. Various standards have been created for the flow rate of air in an operating room with the goal of reducing infectious disease transmission. Laminar airflow is often desirable for this purpose. However, current airflow systems (e.g., comprising air plenums) still produce some amount of turbulent airflow, which allows airborne microbes to travel to and infect a patient despite the moderate to high rate of airflow. Therefore, there remains a need for air plenums that reduce the probability of infectious disease transmission, for example in an operating room.

### SUMMARY

The present invention encompasses, inter alia, the recognition that sharp corners in a cross-section of downward airflow (e.g., as occur when air outlets are arranged in a polygonal shape such as a rectangle) provide regions where laminar flow is disrupted (e.g., turbulence is introduced) and therefore provide an entry point for contaminants into the flow region, which can infect or otherwise harm a patient. Moreover, it is recognized that a constant velocity of air flow across a cross-section can allow airborne contaminants to readily travel through an entire airflow region produced by an air plenum once the contaminants are introduced into the region (e.g., at turbulent corners).

The present invention provides, inter alia, air plenums that produce highly laminar airflow and methods of their use. In certain embodiments, an air plenum produces airflow near and around the perimeter of a downward airflow region (e.g., by entrainment) such that a vertical curtain of air extending down from an outer region near an airflow perimeter is created. Entrainment causes air nearby an outer region of airflow (e.g., ambient air and/or air emanating from a plenum into an inner region of airflow) to be pulled downward and towards the curtain thereby enhancing the effects of the curtain. In certain embodiments, an average airflow (e.g., laminar airflow) velocity in an outer region of airflow is at least 1.5× higher (e.g., at least 2×, at least 3×, at least 4×, or at least 5× higher) than an average airflow velocity of an inner region of airflow (surrounded by the outer region). In certain embodiments, outlets of an air plenum are arranged such that they are bounded by a substantially circular cross section. Some existing air plenums disposed in the ceiling of an operating room are polygonal in shape. In contrast to a polygonal shape with corners, a circular cross section does not have any corners, and it is found that turbulent regions at the perimeter of the downward airflow are eliminated. Such an air curtain can, in certain embodiments, effectively block entry of any airborne contaminant from the penetrating the curtain and travelling into the inner region of airflow (where it could infect or otherwise harm a patient). This blocking mechanism can be useful, for example, in an operating room, where the surgical field must remain highly sterile and the area inside the room (but outside the field) may be less sterile than the surgical field.

In certain embodiments, an air plenum comprises an airflow enclosure comprising one or more vertical bounding surfaces and a horizontal air-output bottom surface, wherein a plurality of air outlets are disposed in the air-output bottom surface. Airflow out of the plurality of air outlets produces an outer region of airflow having an average velocity of airflow (e.g., laminar airflow) that is at least 1.5× higher (e.g., at least 2×, at least 3× higher) than an average velocity of airflow of an inner region of airflow. In certain embodiments, air flows out of an air plenum through a plurality of outlets at least in part due to a pressure gradient between the rotating air in the airflow enclosure and the local environment outside of the air plenum. In certain embodiments, an air plenum comprises an planar interior partitioning member comprising a plurality of interior air outlets that is disposed inside an airflow enclosure of the air plenum. The partitioning member is substantially parallel to, and spatially separated from, the horizontal air-output bottom surface of the airflow enclosure such that a pressure gradient (e.g., a uniform pressure gradient) is provided for outflowing air. A pressure gradient for outflowing air can contribute to the laminar character of the airflow. In certain embodiments, the combination of rotational airflow with higher velocity at an airflow enclosure perimeter than an inner region of the airflow enclosure and a pressure gradient near air outlets disposed in the bottom surface of the airflow enclosure produces downward laminar airflow with a vertical curtain of airflow around an outer region of the airflow at the perimeter.

Certain existing operating rooms have ceiling mounted apparatus, such as surgical lights, that disrupt any downward laminar airflow from the plenum. By contrast, air plenums in accordance with certain embodiments of the present invention have additional features such as recessed housings for mounting surgical lights, thereby reducing the presence of any ceiling mounted systems that could disrupt laminar airflow. Airflow may also act to cool the surgical lights. Housings may also aid in producing rotational flow of air in an airflow enclosure in order to produce a vertical air curtain. In certain embodiments, filtration (e.g., electrostatic air filters or depth filters such as HEPA filters) is located outside the healthcare setting in which a plenum is disposed (e.g., are disposed in portions of inlet air ducts connected to the plenum that are outside of the healthcare setting). It has been found that this can help reduce noise produced by a plenum since a plenum in accordance with certain embodiments itself is very quiet during operation and the noise producing components can be isolated outside of the healthcare setting in which the plenum is disposed. In certain embodiments, a plenum produces noise of no more than 10 dB during normal operation (e.g., when used during a surgery in an operating room) (e.g., no more than 7 dB, no more than 5 dB, or no more than 2 dB).

In one aspect, the present invention is directed to a method of supplying a healthcare setting with airflow using an air plenum, the method comprising: conducting air into the air plenum via one or more inlet air ducts, wherein the air plenum is installed in a ceiling of a healthcare setting and is structured such that the air conducted into the air plenum flows through a plurality of air outlets in the air plenum, wherein the plurality of air outlets are disposed within a bounded cross-sectional area and flowing the air through the plurality of air outlets produces airflow that is substantially downward and laminar, wherein an outer region of the airflow has an average velocity of airflow (e.g., laminar airflow) that is at least 1.5× higher (e.g., at least 2×, at least 2.5×, at least 3×, at least 4×, or at least 5× higher) than an average velocity of airflow (e.g., laminar airflow) of an inner region of the airflow (e.g., wherein the average velocity of airflow in the outer region and the average velocity of airflow in the inner region are each at a plane parallel to the bottom surface or are each a volume average) [e.g., thereby effectively creating a vertical curtain of air extending down from a perimeter of the bounded cross-sectional area (e.g., in the outer region)].

In certain embodiments, the outer region and the inner region share a common center (e.g., are coaxial or concentric) and the common center is a center of the air plenum (e.g., wherein the outer region and the inner region are concentric cylinders located under the bounded cross-sectional area).

In certain embodiments, the air plenum is centered over a patient treatment table. In certain embodiments, the healthcare setting is an operating room and the patient treatment table is an operating table. In certain embodiments, the inner region encompasses the patient treatment table (e.g., such that the patient treatment table is contained within a vertical curtain of air effectively created by the air plenum and extending down from the perimeter of the bounded cross-sectional area).

In certain embodiments, the average velocity of the outer region of the airflow (e.g., laminar airflow) at a height of a (the) patient treatment table is at least 1.5× higher (e.g., at least 2×, at least 2.5×, at least 3×, at least 4×, or at least 5× higher) than the average velocity of the inner region of the airflow (e.g., laminar airflow) at the height of the patient treatment table [e.g., thereby effectively creating a vertical curtain of air extending down from a perimeter of the bounded cross-sectional area (e.g., in the outer region) at the level of the patient treatment table]. In certain embodiments, the average velocity of airflow in the outer region of the airflow is an average of airflow velocity throughout a volume of the outer region and the average velocity of airflow in the inner region of the airflow is an average of airflow velocity throughout a volume of the inner region.

In certain embodiments, (i) the air plenum comprises a planar interior partitioning member and a plurality of interior air outlets that are disposed in the planar interior partitioning member and (ii) the planar interior partitioning member is spatially separated from and substantially parallel to the bounded cross-sectional area such that a pressure gradient is produced when conducting air into the air plenum [e.g., thereby causing (e.g., contributing to) the downward laminar airflow through the plurality of air outlets].

In certain embodiments, the bounded cross-sectional area is circular. In certain embodiments, the bounded cross-sectional area is rectangular.

In certain embodiments, the average velocity of airflow of the outer region of airflow is at least 40 feet per minute (e.g., at least 45 feet per minute, at least 50 feet per minute, or at least 60 feet per minute) and the average velocity airflow of the inner region of airflow is no more than 30 feet per minute (e.g., no more than 25 feet per minute, no more than 20 feet per minute, or no more than 15 feet per minute).

In certain embodiments, the bounded cross-sectional area has a dimension of at least 4 feet (e.g., at least 5 feet, at least 6 feet, at least 7 feet, at least 8 feet, at least 10 feet, at least 12 feet, or at least 15 feet) (e.g., is at least 4 feet, at least 5 feet, at least 6 feet, at least 7 feet, at least 8 feet, at least 10 feet, at least 12 feet, or at least 15 feet in diameter).

In certain embodiments, the air plenum comprises one or more deflectors and each of the one or more inlet air ducts has a respective deflector of the one or more deflectors disposed nearby (e.g., within 3 feet, within 2 feet, or within 1 foot) such that the air conducted through the one or more inlet air ducts is deflected (e.g., redirected) by the one or more deflectors.

In certain embodiments, the one or more deflectors deflect the air conducted into the air plenum via the one or more inlet air ducts such that the air has a velocity in a rotational direction that is substantially parallel to a horizontal air-output bottom surface of the air plenum, wherein the plurality of air outlets are disposed in the horizontal air-output bottom surface (e.g., such that an average velocity of the air is higher near a perimeter of the air plenum than near a center of the air plenum).

In certain embodiments, the air plenum comprises a horizontal baffle (e.g., one or more horizontal baffles) and the method comprises: conducting air into the plenum through one or more inlet air ducts; flowing the air over the horizontal baffle; and flowing the air through the plurality of air outlets after the air flows over the horizontal baffle.

In certain embodiments, the air plenum is an integrated air and lighting plenum. In certain embodiments, one or more lights are each disposed in a respective housing in the integrated air and lighting plenum and the method comprises: cooling the one or more lights by flowing air over the respective housing for each of the one or more lights, wherein the air flows over at least a portion of the respective housing prior to flowing through the plurality of air outlets. In certain embodiments, the respective housing of each of the one or more lights is disposed such that a circle centered at a center of the bounded cross-sectional area intersects a center of the respective housing. In certain embodiments, each of the plurality of air outlets has a circular cross section.

In certain embodiments, the air plenum produces no more than 10 dB of noise (e.g., within the healthcare setting) while the air is conducted into the air plenum and flows through the plurality of air outlets. In certain embodiments, the air plenum produces no more than 2 dB of noise (e.g., within the healthcare setting) while the air is conducted into the air plenum and flows through the plurality of air outlets.

In another aspect, the present invention is directed to an air plenum for disposal in a ceiling of a healthcare setting, the air plenum comprising: an airflow enclosure comprising at least (i) one or more vertical bounding surfaces and (ii) a horizontal air-output bottom surface; one or more inlet air ducts disposed in the one or more vertical bounding surfaces; one or more deflectors, wherein each of the one or more deflectors is disposed near a respective inlet air duct of the one or more inlet air ducts (e.g., within 3 feet, within 2 feet, or within 1 foot) such that air through each of the deflectors is deflected (e.g., redirected) by the respective inlet air duct; a planar interior partitioning member comprising a plurality of interior air outlets, wherein the planar interior partitioning member is spatially separated from and substantially parallel to the horizontal air-output bottom surface; and a plurality of air outlets disposed in the horizontal air-output bottom surface within a bounded cross-sectional area.

In certain embodiments, the air plenum, when disposed in the ceiling, produces downward laminar airflow emanating from the plurality of air outlets when air flows in through the one or more inlet air ducts.

In certain embodiments, the air plenum, when disposed in the ceiling, produces an outer region of airflow (e.g., laminar airflow) and an inner region of airflow (e.g., laminar airflow), wherein the average velocity of the outer region of the airflow (e.g., at a height of a patient treatment table) is at least 1.5×higher (e.g., at least 2×, at least 2.5×, at least 3×, at least 4×, or at least 5× higher) than the average velocity of the inner region of the airflow (e.g., at the height of the patient treatment table) [e.g., thereby effectively creating a vertical curtain of air extending down from a perimeter of the bounded cross-sectional area (e.g., in the outer region) at the level of the patient treatment table].

In certain embodiments, the air plenum is disposed in the ceiling, the one or more deflectors are disposed to deflect air conducted into the air plenum via the one or more inlet air ducts such that the air has a velocity in a rotational direction that is substantially parallel to a horizontal air-output bottom surface of the air plenum.

In certain embodiments, the one or more vertical bounding surfaces is one vertical bounding surface which is a side of a cylinder. In certain embodiments, the one or more vertical bounding surfaces is four bounding surfaces which are each a side of a rectangle. In certain embodiments, the bounded cross-sectional area has a circular perimeter. In certain embodiments, the bounded cross-sectional area has a rectangular perimeter. In certain embodiments, each of the plurality of air outlets has a circular cross section.

In certain embodiments, the air plenum comprises horizontal baffle (e.g., one or more horizontal baffles) disposed (e.g., each disposed) (i) within the airflow enclosure and (ii) below the one or more deflectors and above the plurality of air outlets.

In certain embodiments, the air plenum, when disposed in the ceiling of the healthcare setting, is centered over a patient treatment table. In certain embodiments, the healthcare setting is an operating room and the patient treatment table is an operating table.

In certain embodiments, the air plenum is made up of a plurality of modular components sized and shaped to form an arrangement that defines the airflow enclosure (e.g., and, optionally, the planar interior partitioning member).

In certain embodiments, the air plenum is an integrated air and lighting plenum. In certain embodiments, one or more light housings disposed in the horizontal air-output bottom surface [e.g., wherein each of the one or more light housings comprises a light (e.g., a surgical light disposed therein)] and the plurality of air outlets are disposed at least partially around the one or more light housings. In certain embodiments, the one or more light housings are disposed such that a circle centered at a center of the bounded cross-sectional area intersects a center of each of the one or more light housings.

In certain embodiments, conducting air into the air plenum via one or more inlet air ducts comprises: conducting air through an electrostatic air filter, such that at least a portion of contaminants (e.g., negatively charged microbes) in the air are removed; and subsequently, conducting air through a depth filter (e.g., a HEPA filter).

In certain embodiments, the air plenum, when disposed in the ceiling of the healthcare setting, produces no more than 10 dB of noise (e.g., within the healthcare setting) during operation (e.g., when air is conducted into the air plenum and flows through the plurality of air outlets). In certain embodiments, the air plenum, when disposed in the ceiling of the healthcare setting, produces no more than 2 dB of noise (e.g., within the healthcare setting) during operation (e.g., when air is conducted into the air plenum and flows through the plurality of air outlets).

In another aspect, the present invention is directed to an air plenum for disposal in a ceiling of a healthcare setting, comprising: an airflow enclosure comprising at least (i) one or more vertical bounding surfaces and (ii) a horizontal air-output bottom surface; one or more inlet air ducts disposed in the one or more vertical bounding surfaces; a plurality of air-outlets disposed in the horizontal air-output bottom surface within a bounded cross-sectional area; and a horizontal baffle (e.g., one or more horizontal baffles) disposed (e.g., each disposed) (i) within the airflow enclosure and (ii) below the one or more inlet air ducts and above the plurality of air-outlets.

In certain embodiments, the air plenum, when disposed in the ceiling, produces laminar airflow emanating from the plurality of air-outlets when air flows in through the one or more inlet air ducts.

In certain embodiments, the air plenum, when disposed in the ceiling, produces an outer region of airflow (e.g., laminar airflow) and an inner region of airflow (e.g., laminar airflow), wherein the average velocity of the outer region of the airflow (e.g., at a height of a patient treatment table) is at least 1.5× higher (e.g., at least 2×, at least 2.5×, at least 3×, at least 4×, or at least 5× higher) than the average velocity of the inner region of the airflow (e.g., at the height of the patient treatment table) [e.g., thereby effectively creating a vertical curtain of air extending down from a perimeter of the bounded cross-sectional area (e.g., in the outer region) at the level of the patient treatment table].

In certain embodiments, the bounded cross-sectional area has a circular perimeter. In certain embodiments, the bounded cross-sectional area has a rectangular perimeter. In certain embodiments, each of the plurality of air-outlets has a circular cross section.

In certain embodiments, the one or more vertical bounding surfaces is one vertical bounding surface which is a side of a cylinder. In certain embodiments, the one or more vertical bounding surfaces is four bounding surfaces which are each a side of a rectangle.

In certain embodiments, the air plenum, when disposed in the ceiling of the healthcare setting and centered over a patient treatment table. In certain embodiments, the healthcare setting is an operating room and the patient treatment table is an operating table.

In certain embodiments, the air plenum is made up of a plurality of modular components sized and shaped to form an arrangement that defines the airflow enclosure (e.g., and, optionally, the planar interior partitioning member).

In certain embodiments, the air plenum is an integrated air and lighting plenum. In certain embodiments, the air plenum comprises one or more light housings disposed in the horizontal air-output bottom surface [e.g., wherein each of the one or more light housings comprises a light (e.g., a surgical light disposed therein)] and the plurality of air-outlets are disposed at least partially around the one or more light housings. In certain embodiments, the one or more light housings are disposed such that a circle centered at a center of the bounded cross-sectional area intersects a center of each of the one or more light housings.

In certain embodiments, the air plenum comprises a planar interior partitioning member comprising a plurality of interior air outlets, wherein (i) the planar interior partitioning member is spatially separated from and substantially parallel to the horizontal air-output bottom surface and (ii) the planar interior partitioning member is disposed between the horizontal baffle and the horizontal air-output bottom surface.

In certain embodiments, the air plenum, when disposed in the ceiling of the healthcare setting, produces no more than 10 dB of noise (e.g., within the healthcare setting) during operation (e.g., when air is conducted into the air plenum and flows through the plurality of air outlets). In certain embodiments, the air plenum, when disposed in the ceiling of the healthcare setting, produces no more than 2 dB of noise (e.g., within the healthcare setting) during operation (e.g., when air is conducted into the air plenum and flows through the plurality of air outlets).

In another aspect, the present invention is directed to an airflow system for use in a healthcare setting, the system comprising: an air plenum; an inlet air duct for conducting air into the air plenum; an electrostatic air filter comprising one or more charge plates for producing a positively charged surface; and a depth filter (e.g., a HEPA filter), wherein the electrostatic air filter and the depth filter are disposed on an interior of the inlet air duct and the depth filter is disposed between the electrostatic air filter and the air plenum along a path of air conduction through the inlet air duct.

In certain embodiments, the system comprises a plurality of inlet air ducts (e.g., 2 or 3 or 4 inlet air ducts) for conducting air into the air plenum, wherein each inlet air of the plurality of inlet air ducts has a respective electrostatic air filter and a respective depth filter disposed on an interior of the inlet air duct and the respective depth filter is disposed between the respective electrostatic air filter and the air plenum along a path of air conduction.

In certain embodiments, a removable collection filter is disposed on or near at least one surface of each of the one or more charge plates.

In certain embodiments, the air plenum is disposed in a ceiling of the healthcare setting (e.g., an operating room) and the electrostatic air filter and the depth filter are disposed outside of the healthcare setting (e.g., in an auxiliary room).

In certain embodiments, the air plenum produces no more than 10 dB of noise (e.g., within the healthcare setting) during operation (e.g., when air is conducted into the air plenum and flows out of the air plenum). In certain embodiments, the air plenum, when disposed in the ceiling of the healthcare setting, produces no more than 2 dB of noise (e.g., within the healthcare setting) during operation (e.g., when air is conducted into the air plenum and flows out of the air plenum).

In certain embodiments, the air plenum is any one of the air plenums disclosed herein.

### DEFINITIONS

In order for the present disclosure to be more readily understood, certain terms used herein are defined below. Additional definitions for the following terms and other terms may be set forth throughout the specification.

In this application, the use of “or” means “and/or” unless stated otherwise. As used in this application, the term “comprise” and variations of the term, such as “comprising” and “comprises,” are not intended to exclude other additives, components, integers or steps. As used in this application, the terms “about” and “approximately” are used as equivalents. Any numerals used in this application with or without about/approximately are meant to cover any normal fluctuations appreciated by one of ordinary skill in the relevant art. In certain embodiments, the term “approximately” or “about” refers to a range of values that fall within 25%, 20%, 19%, 18%, 17%, 16%, 15%, 14%, 13%, 12%, 11%, 10%, 9%, 8%, 7%, 6%, 5%, 4%, 3%, 2%, 1%, or less in either direction (greater than or less than) of the stated reference value unless otherwise stated or otherwise evident from the context (except where such number would exceed 100% of a possible value).

In order for the present disclosure to be more readily understood, certain terms used herein are defined below. Additional definitions for the following terms and other terms may be set forth throughout the specification.

In this application, the use of “or” means “and/or” unless stated otherwise. As used in this application, the term “comprise” and variations of the term, such as “comprising” and “comprises,” are not intended to exclude other additives, components, integers or steps. As used in this application, the terms “about” and “approximately” are used as equivalents. Any numerals used in this application with or without about/approximately are meant to cover any normal fluctuations appreciated by one of ordinary skill in the relevant art. In certain embodiments, the term “approximately” or “about” refers to a range of values that fall within 25%, 20%, 19%, 18%, 17%, 16%, 15%, 14%, 13%, 12%, 11%, 10%, 9%, 8%, 7%, 6%, 5%, 4%, 3%, 2%, 1%, or less in either direction (greater than or less than) of the stated reference value unless otherwise stated or otherwise evident from the context (except where such number would exceed 100% of a possible value).

Healthcare environment: As used herein, a healthcare environment is a location where healthcare is given to a patient. In some embodiments, a healthcare environment is a hospital, a clinic, a health emergency facility, an urgent care facility, a doctor's office, or a group of one or more rooms designed for surgery or patient treatment. A setting of a healthcare environment may be a room, a group of rooms, a ward, a department, or a general space in which medical care such as treatments or procedures is administered or performed. In some embodiments, a setting of a healthcare environment is an operating room or operating suite.

Inlet air duct: As used herein, an inlet air duct is an opening or hollow channel through which input air is conducted when in use. An inlet air duct can be, for example, a hole in an airflow enclosure or a metal tube with rectangular or circular cross section (as in a typical HVAC system). In certain embodiments, a metal tube is disposed in an inlet air duct to connect to and guide airflow from an HVAC system. In certain embodiments, an inlet air duct is a metal tube that connects to another tube that is a portion of an HVAC system of a healthcare environment.

Patient treatment table: As used herein, a patient treatment table is any seat, table, or other apparatus on which a patient is positioned (e.g., sits, reclines, or lies) during treatment or in order to receive treatment. In certain embodiments, a patient treatment table is located in a patient exam room or other similar healthcare setting. In certain embodiments, a patient treatment table is an operating table located in an operating room.

Bounded cross-sectional area: As used herein, a bounded cross-sectional area that bounds a plurality of air outlets is a two dimensional area that is the convex hull of the plurality of air outlets. In certain embodiments, a plurality of outlets are disposed in an arrangement such that the bounded cross-sectional area is a polygon (e.g., a rectangle) or a circle. In certain embodiments, a bounded cross-sectional area is tangent to each of a set air outlets in a plurality of air outlets, wherein the set contains each of the outermost air outlets in the plurality of air outlets. For example, a bounded cross-sectional area is an area that includes every region that is between any two air outlets in a plurality of air outlets.

Contaminant: As used herein, a contaminant is a small object that is capable of being airborne. Contaminants reduce the purity (e.g., sterility) of air. In certain embodiments, a contaminant is infectious. For example, a contaminant may be a microbe such as bacteria or another microorganism. In certain embodiments, a contaminant is a particle. For example, a contaminant may be a dust particle. In certain embodiments, a contaminant has a characteristic dimension (e.g., diameter) of less than 10 microns (e.g., less than 7 microns, less than 5 microns, or less than 2 microns).

### BRIEF DESCRIPTION OF THE DRAWINGS

Drawings are presented herein for illustration purposes, not for limitation. Moreover, the drawings are not necessarily to scale. The foregoing and other objects, aspects, features, and advantages of the disclosure will become more apparent and may be better understood by referring to the following description taken in conjunction with the accompanying drawings, in which:

FIG. 1A and FIG. 1B are a perspective and plan view of an exemplary air plenum, according to illustrative embodiments of the disclosure;

FIG. 2 is a plan view of an exemplary integrated air and lighting plenum, according to illustrative embodiments of the disclosure;

FIGS. 3A-3C are views of an exemplary airflow system comprising an air plenum disposed in a ceiling of an healthcare setting and connected to the HVAC system of the corresponding healthcare environment, according to illustrative embodiments of the disclosure;

FIG. 3D is an illustration of an arrangement of an electrostatic air filter and a depth filter disposed in an inlet air duct (e.g., for use in the exemplary airflow system of FIGS. 3A-3C), according to illustrative embodiments of the disclosure;

FIGS. 4A and 4B are photographs of exemplary modular components comprising air outlets that form a horizontal air-output bottom surface and a planar interior partitioning member used in producing a pressure gradient for airflow, according to illustrative embodiments of the disclosure;

FIG. 4C is an illustration of an exemplary modular component that forms a portion of a horizontal air-output bottom surface and a planar interior partitioning member that is spatially separated, each comprising a plurality of air outlets, according to illustrative embodiments of the disclosure;

FIG. 4D is an illustration of an air plenum partially disposed (e.g., being installed) in a ceiling of a healthcare setting wherein one modular component comprising a portion of a horizontal air-output bottom surface and a portion of a planar interior partitioning member is mounted in the ceiling, according to illustrative embodiments of the disclosure;

FIG. 5A is a perspective of a computational fluid dynamics simulation showing rotational airflow in an air plenum comprising deflectors about a plurality of axes perpendicular to the air-output bottom surface of the airflow enclosure as well as downward laminar airflow through a plurality of air outlets, according to illustrative embodiments of the disclosure;

FIG. 5B shows a plan view of only the rotational path of air through the airflow enclosure (air output out of the plurality of air outlets is not shown) corresponding to the perspective view of the exemplary system in FIG. 5A;

FIG. 5C shows airflow in a healthcare setting after the air has flown through the plurality of air outlets in the exemplary plenum shown in FIG. 5A, according to illustrative embodiments of the disclosure;

FIG. 5D shows a cross section of FIG. 5C;

FIG. 5E shows a cross section of a computational fluid dynamics simulation with the air plenum used in FIGS. 5A-5D in an alternate healthcare setting without air return vents at the floor, according to illustrative embodiments of the disclosure;

FIG. 6A shows air velocity of an exemplary air plenum, as calculated using a computational fluid dynamics study and measured six inches below the exemplary air plenum, according to illustrative embodiments of the disclosure;

FIG. 6B shows air velocity of the exemplary air plenum of FIG. 6A measured at a height of an operating table that is centered below the exemplary air plenum (e.g., at a height of approximately 4 feet above the ground); and

FIG. 6C shows a vertical cross section of the airflow from the exemplary air plenum of FIGS. 6A and 6B, wherein the cross section intersects a center of the exemplary air plenum.

### DETAILED DESCRIPTION OF CERTAIN EMBODIMENTS

It is contemplated that systems, devices, methods, and processes of the claimed invention encompass variations and adaptations developed using information from the embodiments described herein. Adaptation and/or modification of the systems, devices, methods, and processes described herein may be performed by those of ordinary skill in the relevant art.

Throughout the description, where articles, devices, and systems are described as having, including, or comprising specific components, or where processes and methods are described as having, including, or comprising specific steps, it is contemplated that, additionally, there are articles, devices, and systems of the present disclosure that consist essentially of, or consist of, the recited components, and that there are processes and methods according to the present disclosure that consist essentially of, or consist of, the recited processing steps.

It should be understood that the order of steps or order for performing certain action is immaterial so long as the invention remains operable. Moreover, two or more steps or actions may be conducted simultaneously.

The mention herein of any publication, for example, in the Background section, is not an admission that the publication serves as prior art with respect to any of the claims presented herein. The Background section is presented for purposes of clarity and is not meant as a description of prior art with respect to any claim. Headers are provided for the convenience of the reader and are not intended to be limiting with respect to the claimed subject matter.

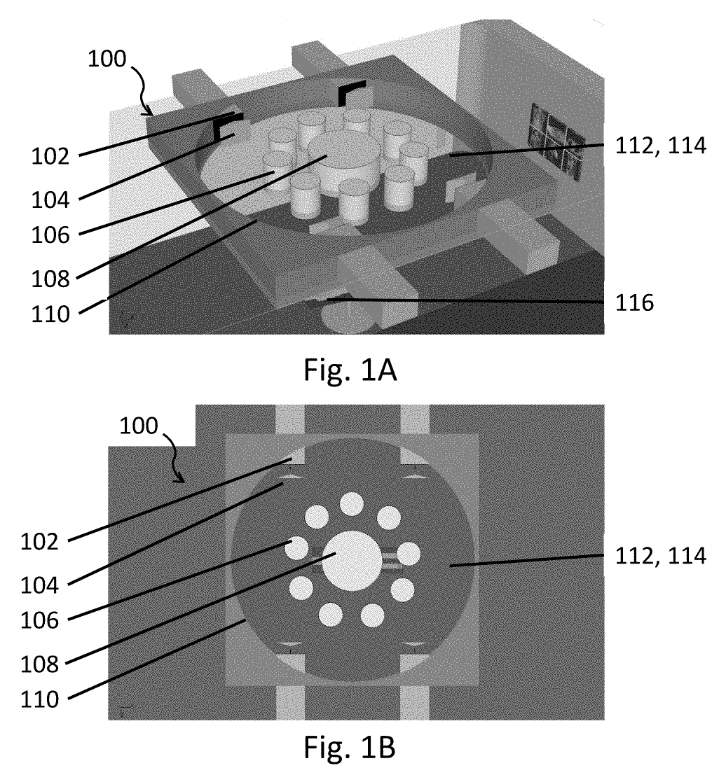

FIGS. 1A and 1B show perspective and plane views, respectively, of an exemplary air plenum 100 according to certain embodiments of the present disclosure. The exemplary air plenum 100 is disposed in the ceiling of a healthcare setting and centered over a patient treatment table 116. Air is conducted into the plenum through four inlet air ducts (e.g., air inlet duct 102). In certain embodiments, an air plenum comprises one or more inlet air ducts. Each inlet air duct has a corresponding deflector 104 disposed near the duct. Conducted air is deflected as it enters the airflow enclosure of plenum. In certain embodiments, a deflector is disposed near an inlet air duct (e.g., within three feet, within two feet, with no more than one foot, or within six inches). Airflow within the plenum is contained by an airflow enclosure comprising one or more vertical bounding surfaces 110 and a horizontal air-output bottom surface 112. Air outlets of the air plenum are disposed in the horizontal air-output bottom surface 112 (and are not shown in FIGS. 1A-1B).

A plurality of housings (e.g., housing 106) are disposed in the airflow enclosure and arranged in a circle around a center point of the airflow enclosure. In certain embodiments, each of a plurality of housings has a light (e.g., a surgical light) disposed therein. A central housing 108 is also disposed in a center of the airflow enclosure. A central housing may have, for example, one or more lights, cameras, microphones, or sensors disposed therein. In certain embodiments, each housing of a plurality of housings and/or a central housing extends up to the top of an airflow enclosure of an air plenum. The plurality of housings and the central housing may modify how air flows through the airflow enclosure.

In certain embodiments, an air plenum comprises a horizontal baffle 114 disposed in a plane substantially parallel (e.g., with less than 15 degrees or less than 10 degrees tilt from parallel) to the bottom surface of an airflow enclosure of an air plenum (e.g., thereby creating a larger pressure gradient driving outward airflow). In certain embodiments, a horizontal baffle (e.g., one or more horizontal baffles) is disposed (e.g., each disposed) (i) within an airflow enclosure and (ii) below one or more deflectors and above a plurality of air outlets (i.e., above a horizontal air-output bottom surface).

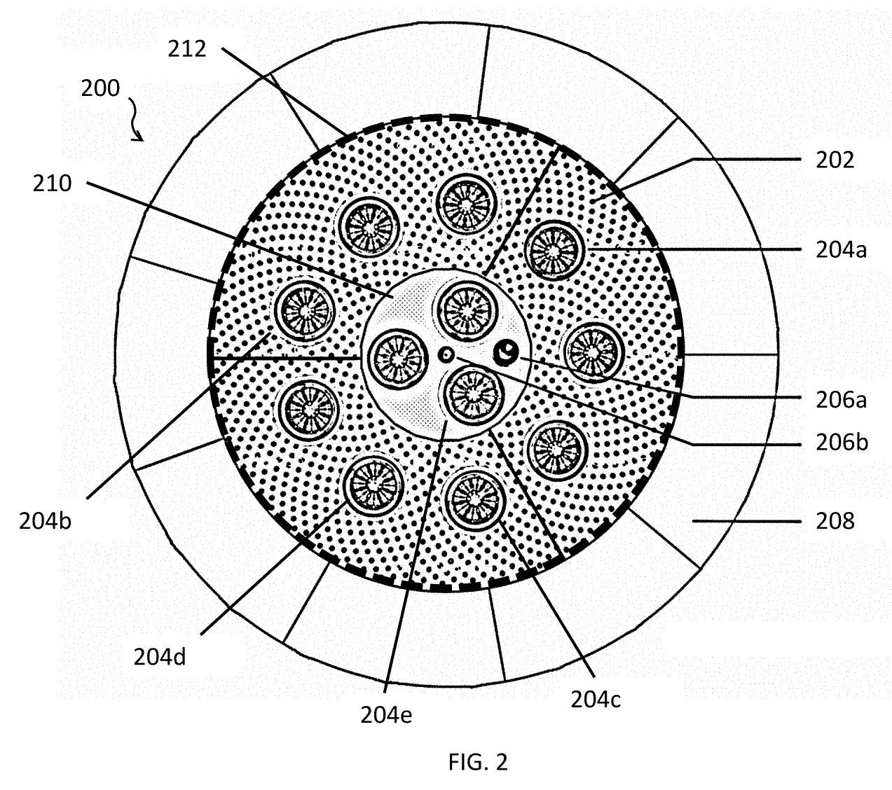

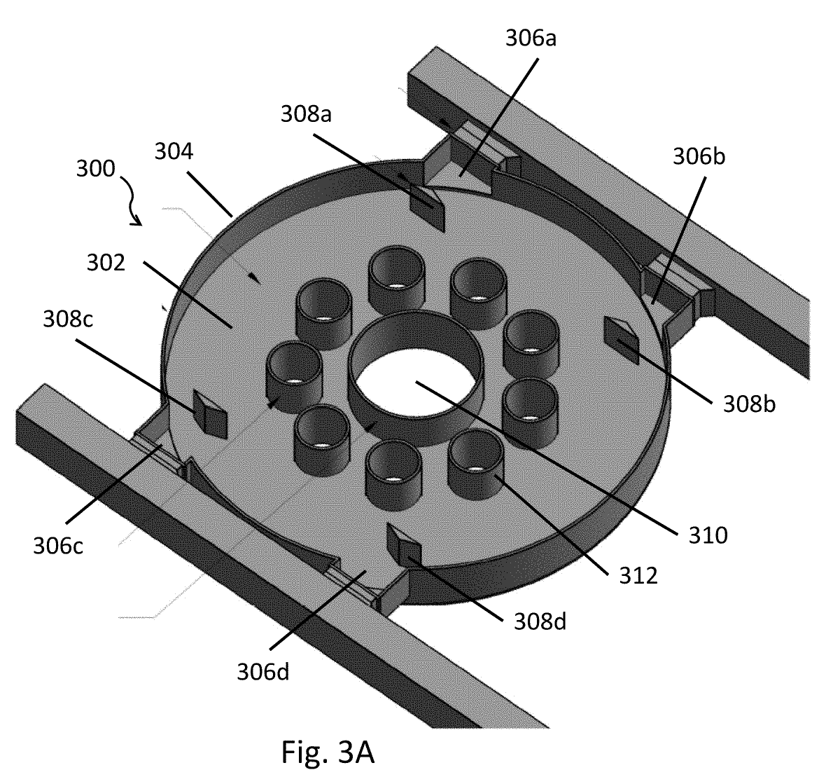

In some embodiments, an air plenum is an integrated air and lighting plenum that is a circular apparatus comprising three concentric units. FIG. 2 shows an exemplary embodiment of an integrated air and lighting plenum 200. Optionally, surfaces of the plenum can be coated or impregnated with formulations of titanium dioxide (TiO₂) that inhibit the growth of bacteria under full-spectrum lighting in order to enhance sterility of the room in which the plenum is located.

A first ring-shaped unit on the periphery of the plenum can be made to include general lighting 208 for the room (e.g., scrub lighting). Such general lighting can provide sufficient, diffuse light necessary for staff to perform necessary functions. The light may be made diffuse by using translucent cover panels for the general illumination light sources. Optionally, the lighting components used for the general lighting may be chosen such that their color is adjustable. The color may be adjusted to the preference of a patient or staff member in order to provide a calming environment or improve visibility for a certain task, for example.

A second ring-shaped unit located interior to the first unit comprises a plurality of surgical lights 204a-c and/or a plurality of housings for surgical lights (not shown in FIG. 2). Such surgical lights or housings for surgical lights may be arranged in a circle having a diameter of up to 84″. In order to provide laminar air flow from the ceiling to the room in which the plenum is located in accordance with HVAC requirements for healthcare environment settings, the second ring-shaped unit may comprise a plurality of airflow outlets 202. Having many surgical lights located in the arrangement allows multiple surgical lights (e.g., 204a, 204b, and 204c) to be used in a coordinated manner (e.g., as a system) to illuminate a patient or work area while also providing redundancy as well as the ability to tailor illumination to fit particular needs (e.g., by coordinating more surgical lights (e.g., 204d)).

A third unit 210 (e.g., central housing) located interior to the second ring-shaped unit comprises additional surgical lights 204e or housings for surgical lights (not shown in FIG. 2) to increase the range of lighting orientations achievable by the plenum in order to satisfy the needs of staff in lighting a surgical site or work area. The surgical lights or housings for surgical lights of the third unit may be arranged in a circle having a diameter of up to 26″. Additionally, such an integrated air and lighting plenum may further comprise one or more accessories such as a webcam 206a, a camera 206b, a microphone, sensors or speakers, to provide additional functionality to the plenum, for example, for monitoring a room or patient or to assist in controlling the surgical lights.

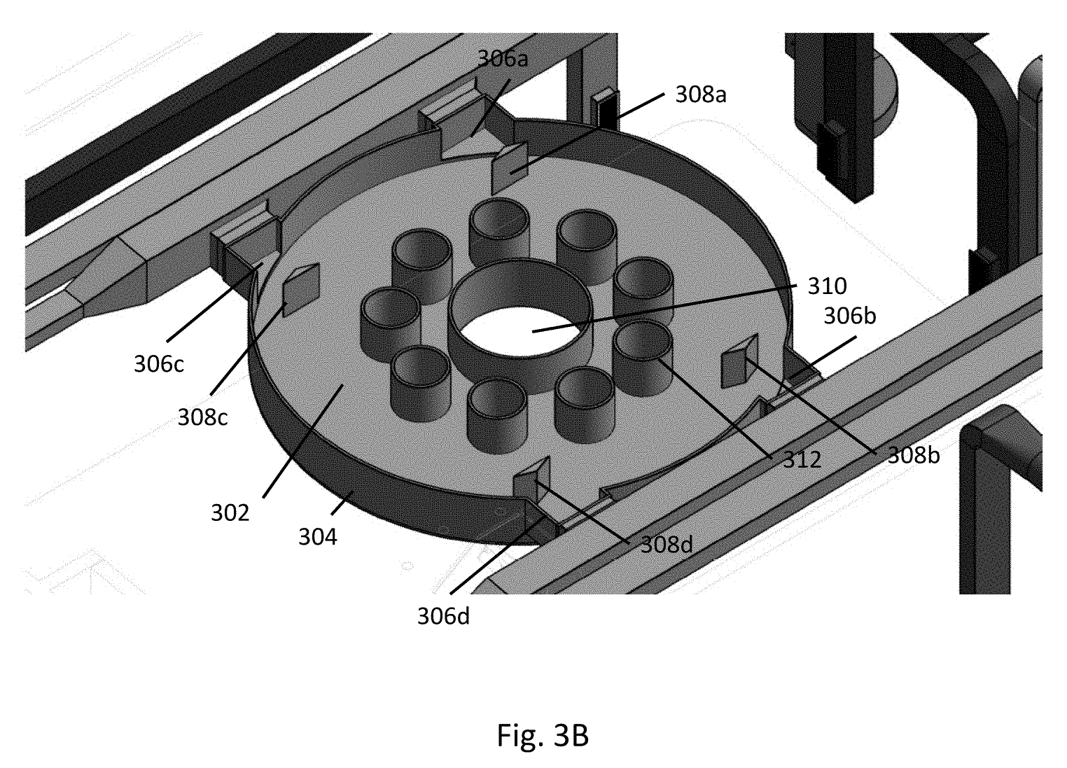

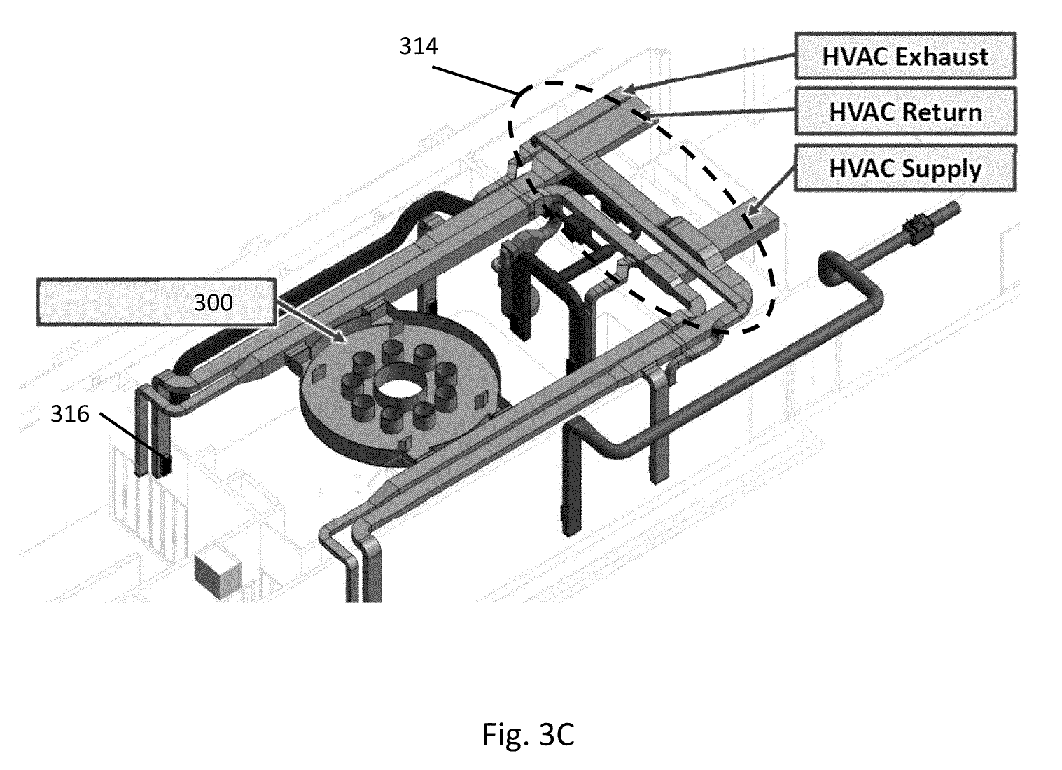

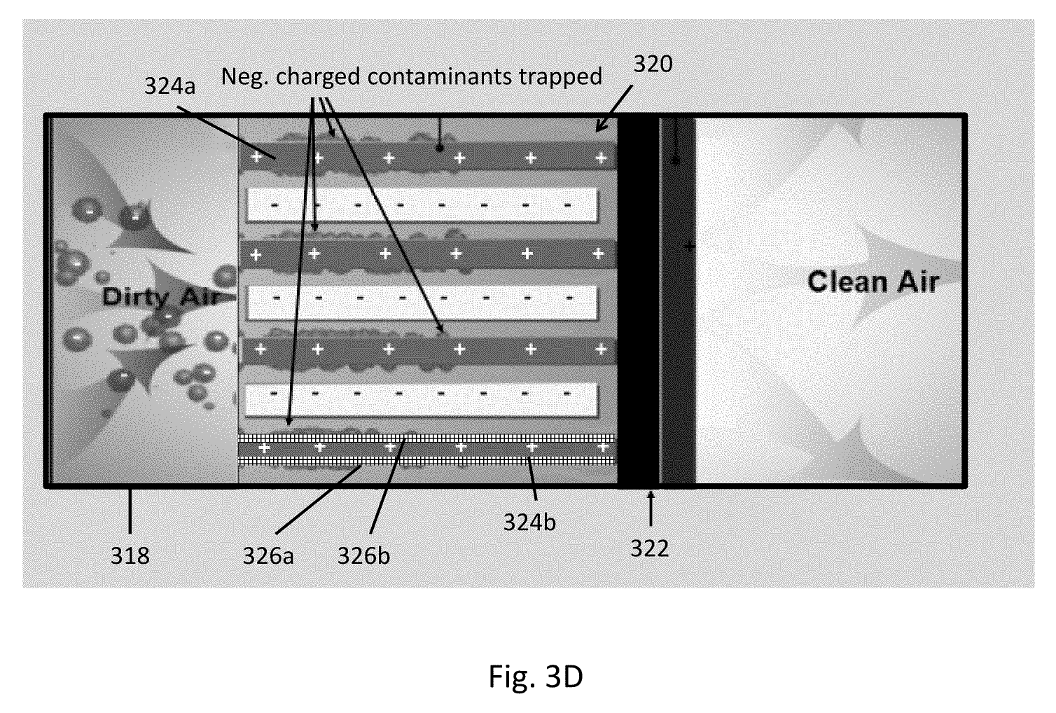

FIG. 3A shows a perspective view of a cross-sectional cut-away of an exemplary air plenum 300. The air plenum comprises four inlet air ducts 306a-d which are connected to an HVAC system of a healthcare environment. The inlet air ducts are disposed in a vertical bounding surface 304 of an airflow enclosure of the air plenum. The airflow enclosure comprises the vertical bounding surface 304 and a horizontal air-output bottom surface 302. In certain embodiments, an air plenum comprises one or more inlet air ducts (e.g., two inlet air ducts, three inlet air ducts, four inlet air ducts, or more). Each of the four inlet air ducts 306a-d has a deflector 308a-d disposed nearby (e.g., within 4 feet, within 3 feet, within 2 feet, or within 1 foot of the output end of the duct). In certain embodiments, inlet air duct(s) are disposed non-perpendicularly to an airflow enclosure of an air plenum (e.g., tangentially or at an obtuse angle) such that air flows first along a perimeter of an airflow enclosure (e.g., along an interior of a vertical bounding surface of the airflow enclosure), thereby having a rotational velocity parallel to a horizontal bottom surface without using a deflector (although one or more deflectors and/or baffle may optionally be used). The air plenum comprises a plurality of air outlets in the horizontal air-output bottom surface (not shown). The airflow enclosure has a circular cross-section. FIG. 3B is an alternative view of the air plenum of FIG. 3A showing the air plenum disposed in a healthcare setting and connected to an HVAC system.

Air plenum 300 has a plurality of housings (e.g., housing 312) arranged in a circular arrangement and a central housing 310. An air plenum may be an integrated air and lighting plenum (e.g., comprising one or more surgical lights disposed therein). An integrated air and lighting plenum may reduce the amount of space occupied by airflow and lighting systems in a healthcare setting. Additionally, lights being disposed within housings in a plenum may reduce disruptions to laminar airflow emanating from the air plenum that would otherwise occur.

In certain embodiments, an air plenum comprises an airflow enclosure with a characteristic dimension (e.g., diameter or side length) of at least 10 feet (e.g., at least 12 feet, at least 14 feet, at least 16 feet, or more). In certain embodiments, a plurality of outlets are bounded by a cross-sectional bounded area with a characteristic dimension (e.g., diameter or side length) of at least 8 feet (e.g., at least 10 feet, at least 12 feet, at least 14 feet, at least 16 feet or more). In certain embodiments, a bounded cross-sectional area has a dimension of at least 4 feet (e.g., at least 5 feet, at least 6 feet, at least 7 feet, at least 8 feet, at least 10 feet, at least 12 feet, or at least 15 feet) (e.g., is at least 4 feet, at least 5 feet, at least 6 feet, at least 7 feet, at least 8 feet, at least 10 feet, at least 12 feet, or at least 15 feet in diameter). In certain embodiments, an airflow enclosure has a circular cross-section such that it has one vertical bounding surface. In certain embodiments, an airflow enclosure has a rectangular cross-section such that it has four vertical bounding surfaces. In certain embodiments, an airflow enclosure is modular such that the airflow enclosure is formed when the pieces are assembled together [e.g., each of one or more vertical bounding surfaces and a horizontal air-output bounding surface comprises one or more modular components attached (e.g., fastened) together].

Referring to FIG. 3C, the HVAC supply, return, and exhaust of the healthcare setting are shown in relation to the exemplary air plenum 300 disposed in the ceiling. Air return vents (e.g., vent 316) near the floor of the healthcare setting can reduce air recirculation in the healthcare setting. In certain embodiments, filtration (e.g., electrostatic air filters and/or depth filters such as HEPA filters) are located outside the healthcare setting in which a plenum is disposed (e.g., are disposed in portions of HVAC inlet air ducts connected to the plenum that are outside of the healthcare setting). Filters can be disposed within portions of ducts that are in an auxiliary room to a healthcare setting in which an air plenum is disposed (e.g., as indicated by dashed circle 314 in FIG. 3C). This can help reduce noise produced by a plenum. In certain embodiments, a plenum produces noise of no more than 10 dB during normal operation (e.g., when used during a surgery in an operating room) (e.g., no more than 7 dB, no more than 5 dB, or no more than 2 dB). Disposing air filters outside of the walls of a healthcare setting can greatly reduce the noise produced by the system (HVAC system and plenum). In conventional air handling systems for healthcare settings, filters are disposed very near a plenum, which produces disruptive noise. As shown in FIG. 3C by the dashed circle, such filters are, in this example, disposed in an auxiliary room that is separated by a wall from the healthcare setting where the air plenum is disposed.

In certain embodiments, charged plates (e.g., inside an electrostatic air filter) are disposed on the interior (e.g., one or more interior sides) of an HVAC inlet air duct and are disposed before any filters present along an airflow path of input air. In certain embodiments, such charged plates can collect contaminants, which are typically negatively charged, to prevent their entry into an air plenum, thereby preventing contamination of an inner region of airflow around a patient treatment table (e.g., which is inside of a vertical curtain formed around an outer region that surrounds the inner region) with contaminants that may otherwise be in the input air. Putting an electrostatic air filter before a depth filter (e.g., HEPA filter) may reduce noise and/or prolong filter lifetime by removing contaminants before they contact the filter.

FIG. 3D shows an exemplary arrangement of an electrostatic air filter 320 disposed before a depth filter 322 (e.g., a HEPA filter) in an inlet air duct 318. Negatively charged contaminants are trapped (e.g., adsorbed) on surfaces of positively charged plates (e.g., plates 324a-b) of the electrostatic air filter 320 as air flows over the filter. Because many biological contaminants like microbes are naturally negatively charged due to a negative charge of their cell walls or membranes, a separate ionizer is not necessarily needed. An ionizer may otherwise introduce risk associated with charged air in a healthcare setting. In certain embodiments, one or more removable filters 326a-b are disposed on or near the positively charged surfaces of a charged field filter such that the positively charged surfaces attract negatively charged contaminants (e.g., naturally negatively charged contaminants) which are trapped (e.g., adsorbed) onto the one or more removable collection filters. The collection filter(s) can be removed and replaced or removed and cleaned (e.g., sterilized) and replaced, for example.

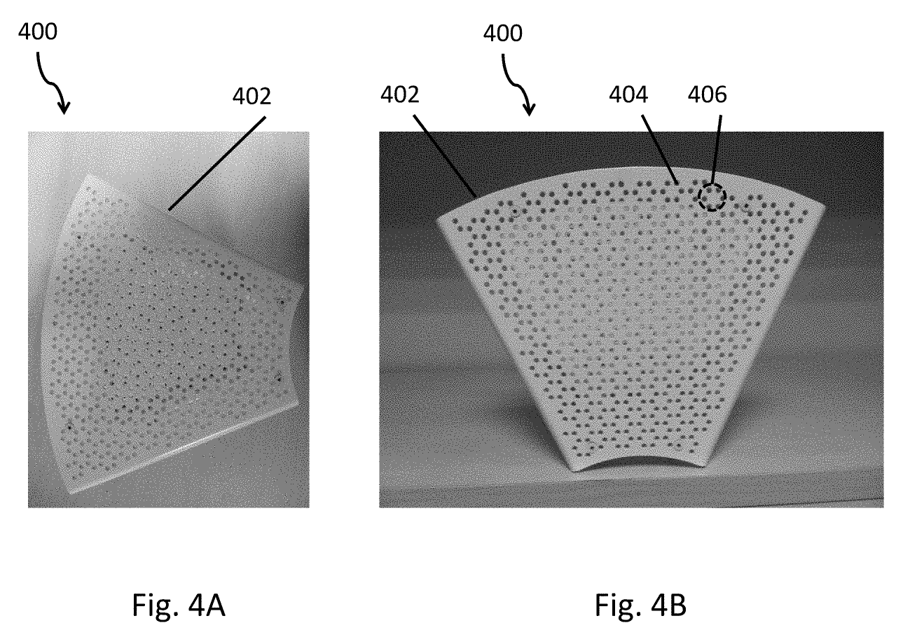

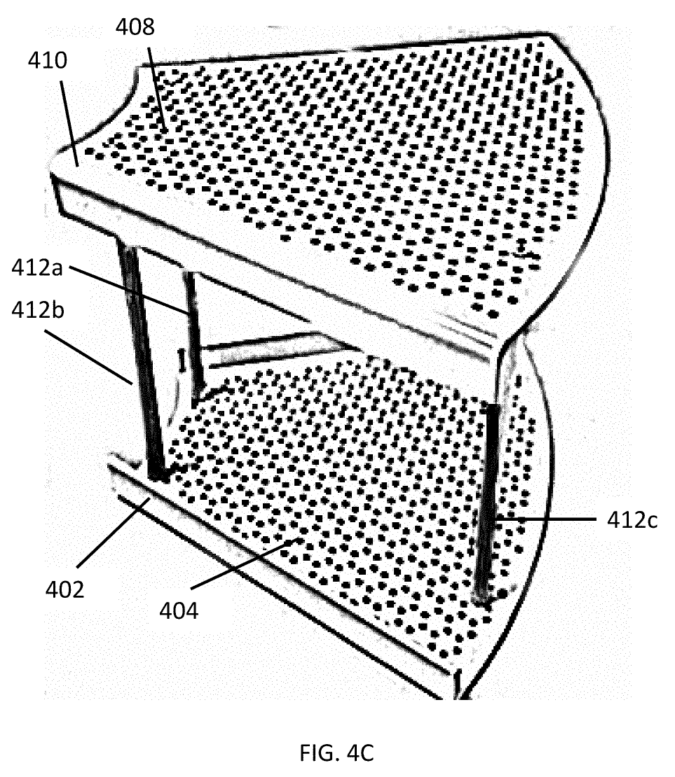

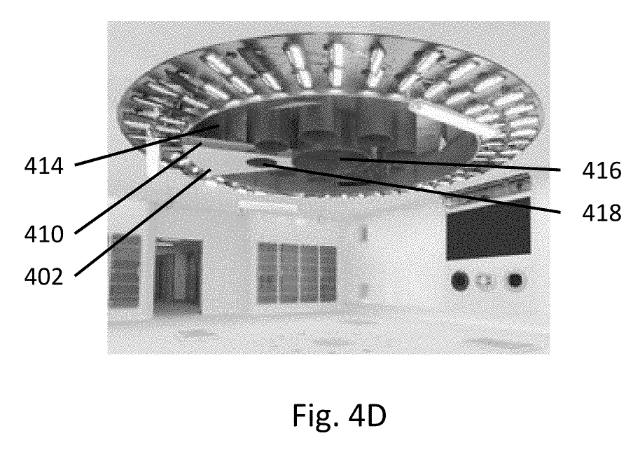

FIGS. 4A and 4B show two photographs of an exemplary modular component 400 that can be used in an air plenum to form a horizontal air-output bottom surface 402 of an airflow enclosure. In certain embodiments, an air plenum comprises two layers of air outlets: one layer in the horizontal air-output bottom surface of the airflow enclosure and one layer disposed in an interior partitioning member that is substantially parallel (e.g., with less than 15 degrees or less than 10 degrees tilt from parallel) to and spatially separated from the horizontal bottom surface. Such an arrangement can produce a pressure gradient for outflowing air from the air plenum. The modular components shown in FIGS. 4A and 4B each have a portion of an interior partitioning member (partially visible behind a portion of a horizontal air-output bottom surface) in addition to a portion of a horizontal air-output bottom surface of an airflow enclosure 402. Whether modular or not, a horizontal air-output bottom surface comprises a plurality of air outlets 404.

In certain embodiments, each air outlet of a plurality of air outlets is circular (i.e., having a circular cross-section) (e.g., such that it defines a cylindrical hole in the bottom surface of an airflow enclosure). In certain embodiments, a plurality of air outlets are disposed in an array of circular sub-arrangements (e.g., sub-arrangement 406). In certain embodiments, a plurality of air outlets are each circular and are also disposed in an array of circular sub-arrangements. The use of circular air outlets and/or an array of circular sub-arrangements can promote improved laminar flow out of an air plenum (e.g., versus rectangular air outlets and/or a rectangular lattice) by reducing turbulent flow.

In certain embodiments, a plurality of air outlets disposed in a bottom surface of an airflow enclosure of an air plenum are bounded (e.g., disposed) within a cross-sectional bounding area that is circular. In certain other embodiments, a plurality of air outlets disposed in a bottom surface of an airflow enclosure of an air plenum are bounded (e.g., disposed) within a cross-sectional bounding area that is rectangular. In certain embodiments, a cross-sectional bounding area within which a plurality of air outlets of an air plenum are disposed has the same shape (e.g., circular or rectangular) as a cross-section of an airflow enclosure of the air plenum. A circular cross-sectional bounding area eliminates corners from the cross section of downward airflow, thereby producing more uniform laminar airflow (e.g., reducing or eliminating regions of turbulent flow).

FIG. 4C is an illustration of a modular component for use in an air plenum. The modular component forms both a portion of a horizontal bottom surface of an airflow enclosure 402 and a portion of an interior partitioning member 410. The portion of the horizontal air-output bottom surface 402 has a plurality of air outlets 404 disposed therein. The portion of the interior partitioning member 410 has a plurality of interior air outlets 408 disposed therein. One or more support members 412a-c attach and separate the portion of the interior partitioning member to the portion of the horizontal air-output bottom surface. For example, the support members are bolted, or otherwise fastened, to each of the member and the bottom surface of the airflow enclosure.

In general, both a horizontal air-output bottom surface of an airflow enclosure and an interior partitioning member may be separately and/or independently modular. For example, modular portions of a horizontal air-output bottom surface are not necessarily attached to modular portions of an interior partitioning member. For example, both or one or neither of a horizontal air-output bottom surface and an interior partitioning member are modular. In certain embodiments, a horizontal air-output bottom surface is a single piece comprising a plurality of air outlets disposed therein. In certain embodiments, an interior partitioning member is a single piece comprising a plurality of interior air outlets disposed therein.

FIG. 4D shows an exemplary modular component disposed in a ceiling during installation of an air plenum. As can be seen from this view, once fully installed and operational, air will be conducted into the airflow enclosure of the air plenum and flow first through the plurality of interior air outlets disposed in the interior partitioning member 410 and then flow through the plurality of air outlets disposed in the horizontal air-output bottom surface 402 of the airflow enclosure 414 of the plenum. The spatial separation between the interior partitioning member 410 and the horizontal air-output bottom surface 402 forms a pressure gradient that results in substantially downward laminar airflow as air conducted into the plenum through one or more inlet air ducts flows out of the plenum. In this example, the air plenum comprises a plurality of housings (e.g., housing 418) disposed in a circular arrangement centered around a center of the airflow enclosure and each modular component forming a portion of the horizontal air-output bottom surface comprises an opening in which a portion of one of the plurality of housings is disposed (e.g., housing 418). In certain embodiments, as shown in FIG. 4D modular components form a ring around a central housing 416. In certain embodiments, a horizontal baffle is disposed below one or more deflectors and above a planar interior partitioning member of an air plenum (e.g., or at least above a horizontal air-output bottom surface) (e.g., and around any housings present).

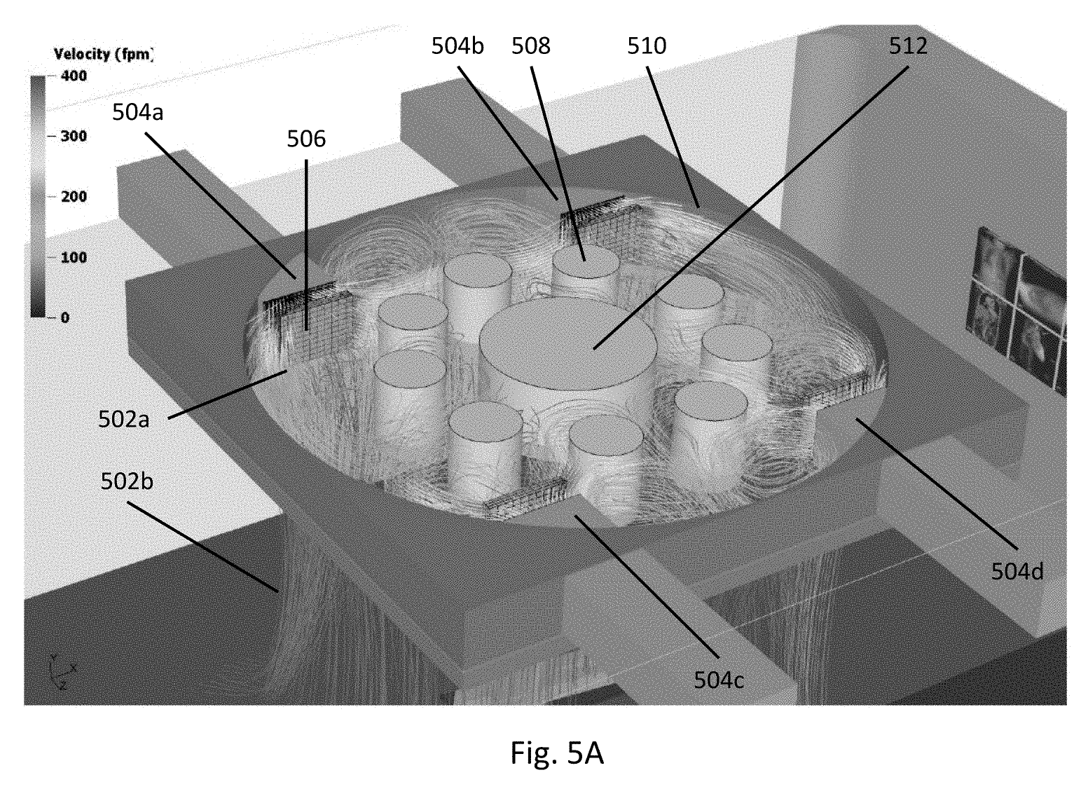

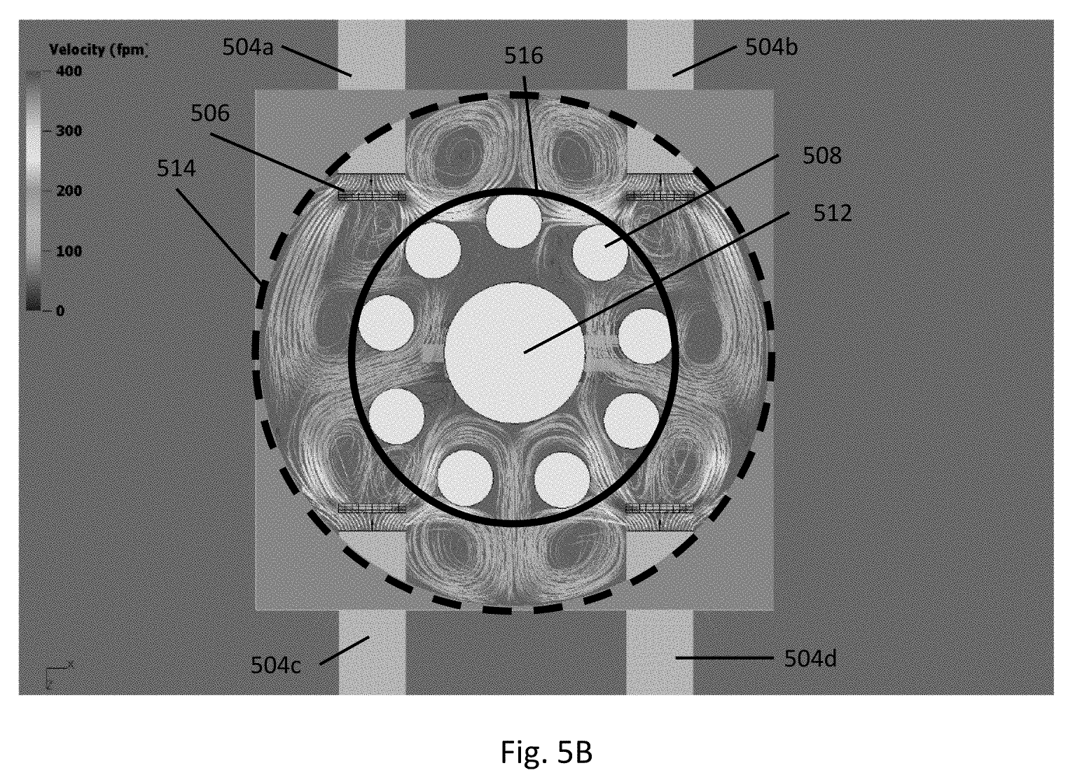

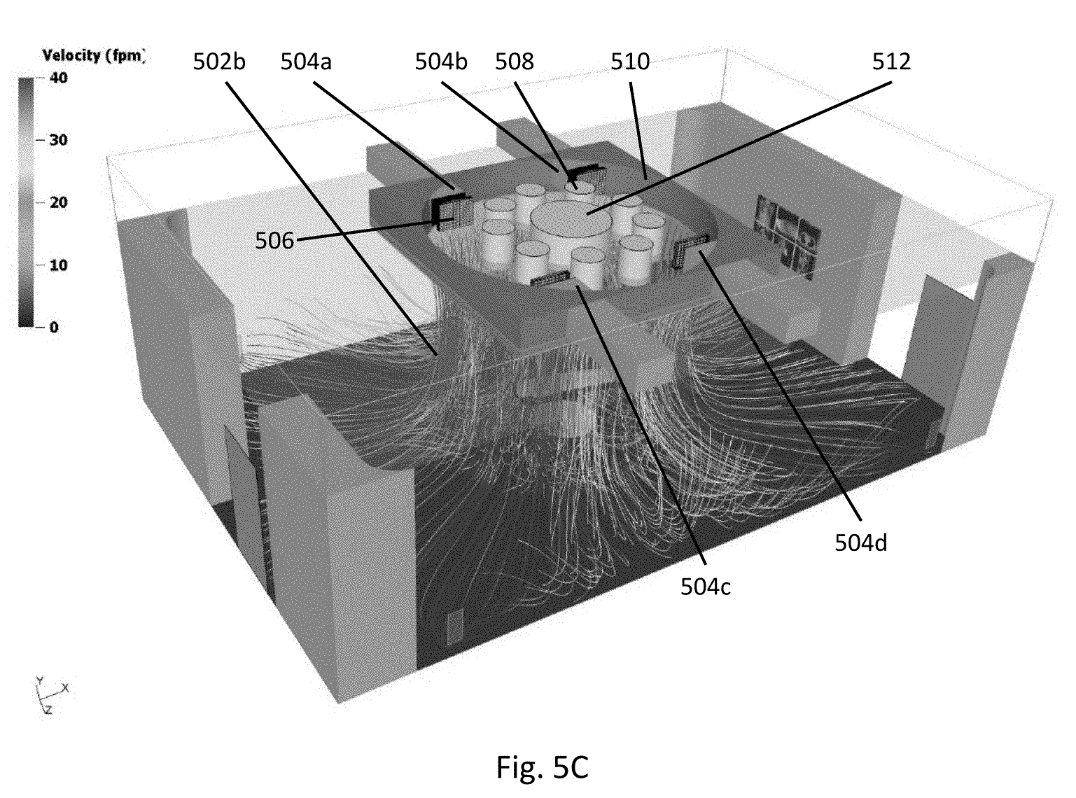

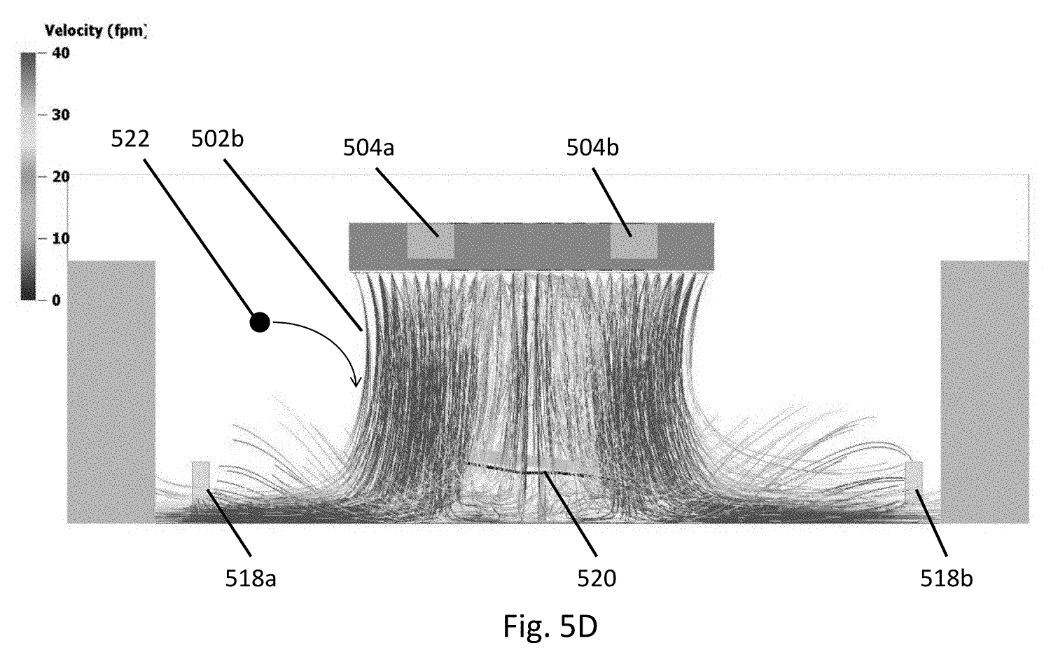

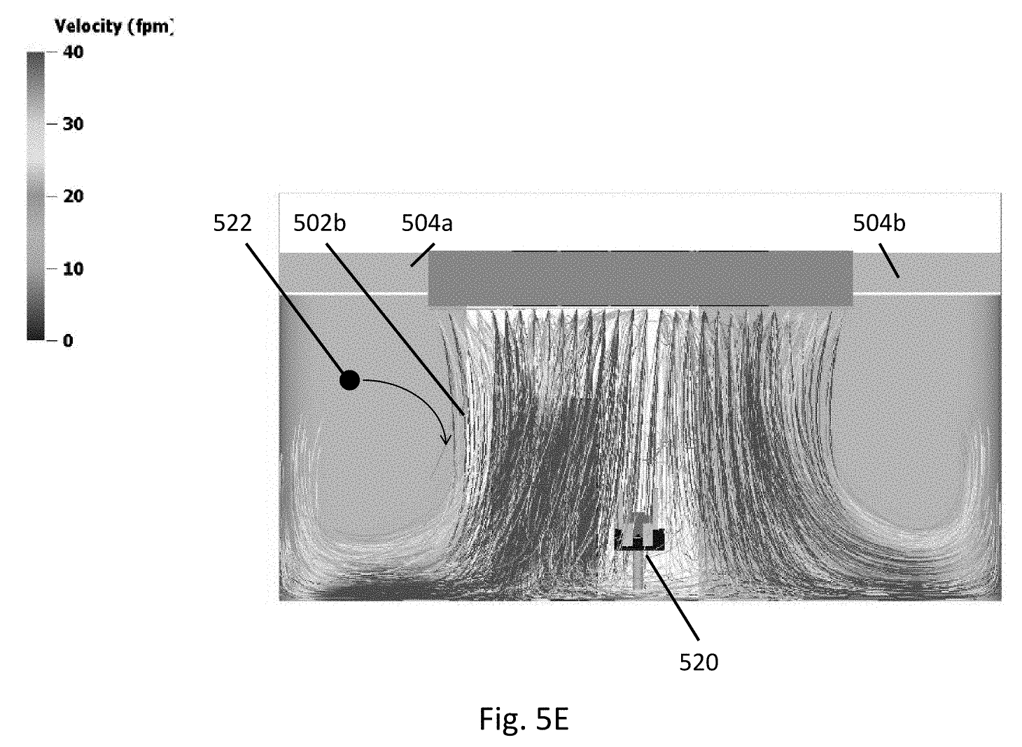

FIG. 5A shows a computational fluid dynamics simulation of airflow through an air plenum disposed in a healthcare setting in accordance with certain embodiments of the present disclosure (e.g., an air plenum in accordance with FIGS. 3A and/or 4D). Air is conducted through one or more inlet air ducts (four inlet air ducts 504a-d as shown). In some embodiments, as shown in the exemplary embodiment shown in FIG. 5A, a deflector 506 is disposed near each inlet air duct (e.g., within 3 feet, within 2 feet, or within 1 foot) and air conducted through the inlet air ducts 502a is deflected by the deflector 506. Deflected air [e.g., all or substantially all of the air conducted into the airflow enclosure of the plenum (e.g., >90%)] has a velocity in a rotational direction that is substantially parallel to the horizontal air-output bottom surface of the air plenum, as shown by the individual flow lines in FIG. 5A. In other words, air flows in one or more swirls throughout the airflow enclosure parallel to the bottom surface. In certain embodiments, airflow close to a vertical bounding surface 510 of an airflow enclosure has a higher average velocity than airflow on an interior of the enclosure (e.g., near or nearer the center of the enclosure). The velocity of the air has a rotational direction causing it to rotate about one or more axes perpendicular to the horizontal air-output bottom surface of the plenum. Air subsequently flows through a plurality of air outlets in the horizontal air-output bottom surface of the plenum thereby providing substantially downward laminar airflow 502b to the healthcare setting.

A portion of air may also have a rotational velocity such that it rotates around each of a plurality of housings (e.g., housing 508) while moving through the airflow enclosure. This can act to cool a component disposed in the housings (e.g., surgical lights) by convection, which may be beneficial for the component (e.g., improve component operational lifetime). Referring still to FIG. 5A, air cannot flow out of any of the plurality of housings (e.g., housing 508) or the central housing 512 thereby reducing the amount of air as well as the average velocity of airflow of an inner region of the airflow enclosure as well as the average velocity of airflow flowing out of an inner region of the horizontal air-output bottom surface of the plenum. In certain embodiments, there are no air outlets in a central region of a horizontal air-output bottom surface of an airflow enclosure (e.g., in place of a central housing 512) thereby preventing air from flowing out of the central region.

FIG. 5B shows a plan view of FIG. 5A. As can be seen in FIG. 5B, deflectors (e.g., deflector 506) cause a larger portion of high velocity air to flow in a rotational direction in an outer region 514 of the airflow enclosure near the perimeter (the vertical bounding surface 510) than the portion of high velocity air flowing in an inner region 516 (e.g., surrounded by the outer region). The outer region 514 is bounded on its perimeter by the dashed circle and on its interior by the solid circle, while the inner region 516 is bounded on its perimeter by the solid circle. Therefore, an average air velocity in the airflow enclosure is higher in the outer region than in the inner region. FIG. 5C shows a perspective view of air flowing out of the plurality of air outlets in the horizontal air-output bottom surface of the plenum. The substantially downward airflow 502b is highly laminar and has higher average velocity in an outer region (e.g., corresponding to the outer region of the airflow enclosure with higher average velocity in a rotational direction) than in an inner region (e.g., corresponding to the inner region of the airflow enclosure), thereby effectively creating a vertical curtain of air (e.g., due to entrainment of air near the outer region). A pressure gradient between the interior of the airflow enclosure of the plenum and the area of the healthcare setting near the exterior of the plenum forces air to flow out of the plenum at an appreciable velocity (e.g., no less than about 10 feet per minute (fpm)). The use of two layers of air outlets (e.g., in an interior partitioning member and a horizontal air-output bottom surface) can create a sufficient pressure gradient to cause highly laminar airflow (e.g., wherein substantially all airflow out of the plenum is laminar).

FIG. 5D shows an additional cross-sectional view of the air outflow shown in FIG. 5C. As is exemplified in FIG. 5D, contaminants (e.g., contaminant 522) outside of the vertical curtain of air are redirected downward if they flow near to the downward laminar airflow 502b produced by the plenum as they cannot penetrate the vertical curtain that has been formed. The contaminant is not drawn to scale. An airflow system (e.g., to which the air plenum is connected) of the healthcare setting shown for which the simulation was run comprises one or more air return vents 518a-b disposed near the floor of the healthcare setting (e.g., at a height of no more than three feet above the floor). The air plenum is disposed over a patient treatment table 520.

The air return vents 518a-b allow airflow out of the healthcare setting, thereby reducing recirculation of air within the healthcare setting. Reduced recirculation may improve effectiveness of a vertical air curtain in preventing contamination of an inner region under an air plenum. A fan may be disposed inside ducts connected to air return vents in order to readily allow airflow out of a healthcare setting in order to further reduce recirculation of air in the healthcare setting (e.g., beyond what is possible with an air return vent alone). Return vents are also shown in FIG. 3C. Results for an alternative healthcare setting without air return vents are shown in FIG. 5E, where the floor and walls of the healthcare setting cause recirculation of air in the outer perimeter of the room.

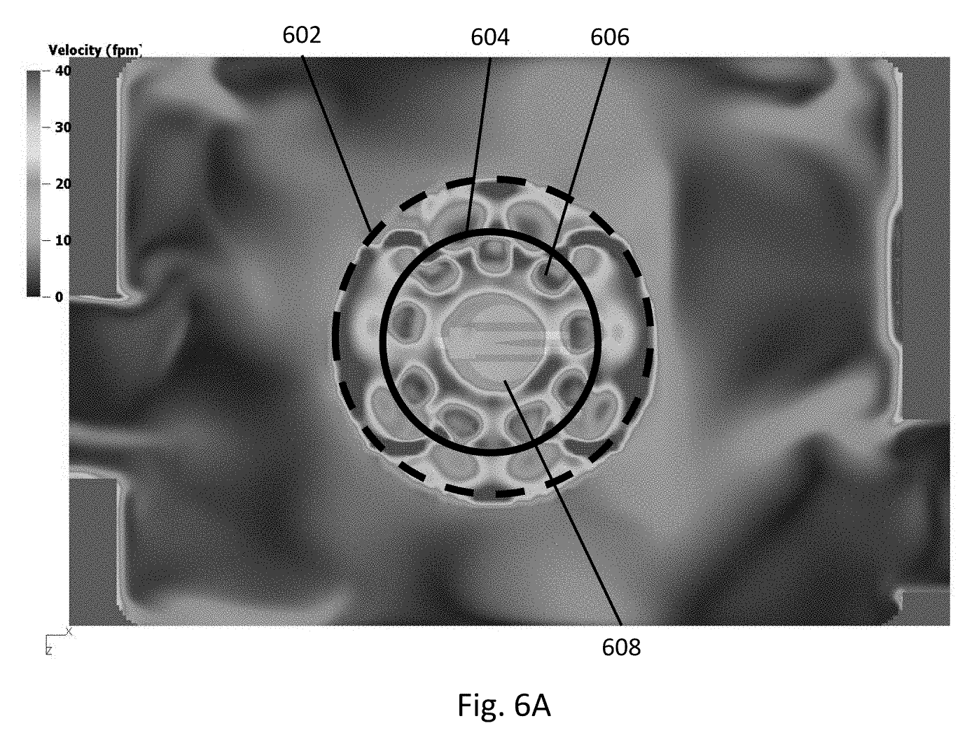

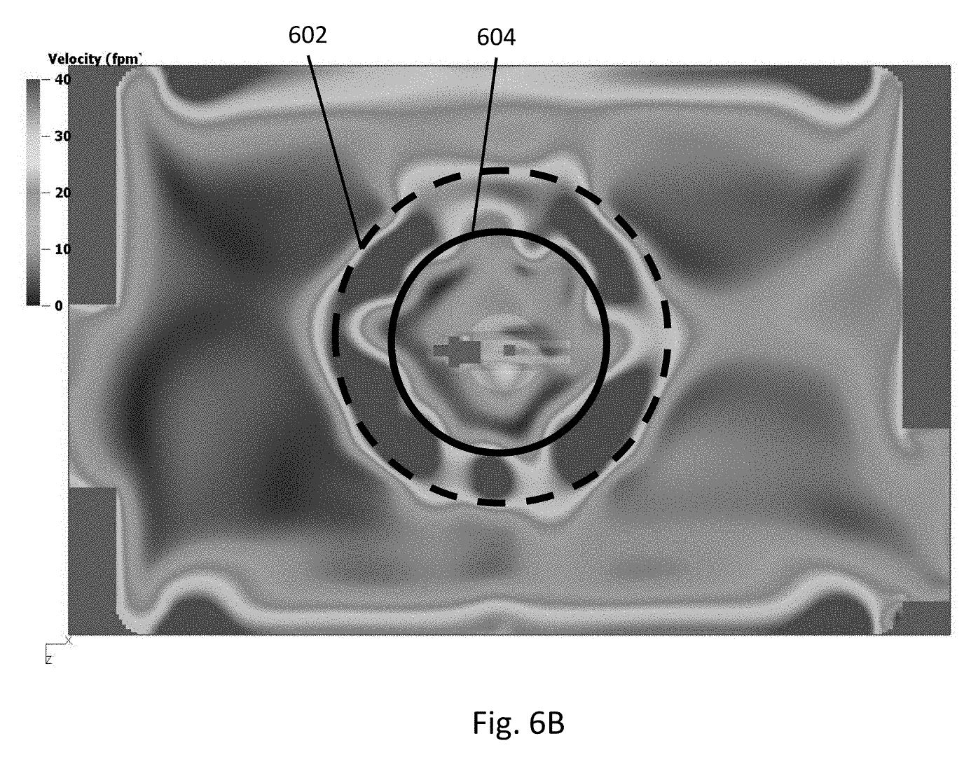

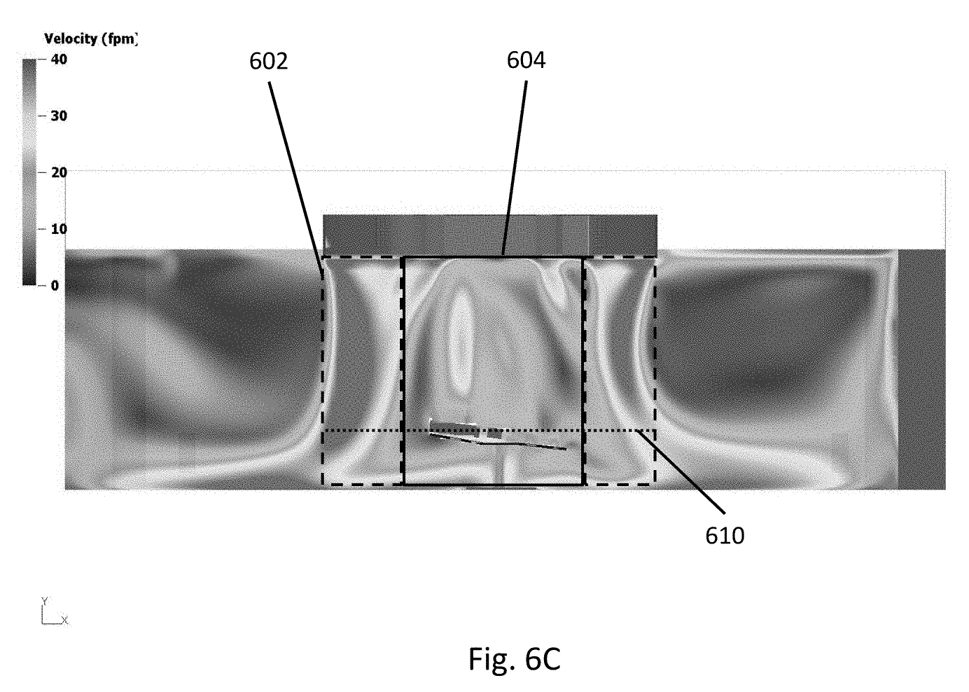

FIG. 6A shows a cross-sectional air velocity profile for a computational fluid dynamics simulation of an air plenum in accordance with certain embodiments of the present disclosure (e.g., an air plenum in accordance with FIGS. 3A and/or 4D). The cross-section is taken at approximately six inches below the horizontal air-output bottom surface of the plenum. An average velocity of airflow of an outer region of airflow 602 is high and an average rate of airflow of an inner region of airflow 604 is low. The outer region is bounded in cross-section by the dashed circle on its perimeter and the solid circle in and the inner region is bounded in cross-section by the solid circle. Therefore, the outer region 602 and inner region 604, in this exemplary embodiment at least, are concentric cylinders (i.e., extending down to the floor of the healthcare setting). An average velocity of airflow immediately under each of the plurality of housings 606 and the central housing 608 of the exemplary plenum is very low (e.g., <10 fpm, close to 0 fpm). A patient treatment table is centered under the plenum and encompassed by the inner region.

FIG. 6B shows a parallel cross-section to that of the cross-section shown in FIG. 6A taken at the level of a patient treatment table centered under the plenum (e.g., approximately 3-5 feet above a floor of the healthcare setting in which the air plenum is disposed). Without wishing to be bound to any particular theory, due to an entrainment effect, a higher average velocity of airflow in the outer region 602 causes nearby air (e.g., ambient air and air emanating into the inner region from the air plenum) to be entrained such that, in effect, a vertical curtain of air is formed. The curtain is an outer region 602 of appreciably higher average velocity of airflow than (i) an average velocity of airflow of an inner region 604 that is surrounded by the outer region 604 and (ii) an average velocity of airflow in the healthcare setting outside of the outer region (e.g., ambient airflow in the setting). Air (e.g., flowing air) from an inner region or ambient air can be entrained. For example, air outlets above an inner region of airflow produced by an air plenum may produce relatively high velocity air (e.g., as shown in regions immediately surrounding the housings shown in FIG. 6A) that is entrained into an outer region, thereby forming a curtain in the outer region of airflow and creating an appreciable difference in average velocity of airflow between the outer region and the inner region (e.g., at least a 1.5× difference at a lower height, as seen in FIG. 6B).

As used herein, an inner region of airflow and an outer region of airflow produced by airflow out of a plurality of air outlets are regions between the horizontal air-output bottom surface of an airflow enclosure of an air plenum and the floor of the healthcare setting in which the plenum is disposed. In certain embodiments, a region is a volume formed by a portion of a bounded cross-sectional area. In certain embodiments, an outer region and an inner region form concentric cylinders. In certain embodiments, a region extends from the floor of a healthcare setting to the horizontal air-output bottom surface of an air plenum. An average velocity of airflow in a region can be an average throughout the volume of a region or an average within a cross-section at a certain height [e.g., the height of a patient treatment table (e.g., approximately 3 feet or 4 feet above the floor of a healthcare setting or in a range of 3 to 5 feet for a table with adjustable height)].

FIG. 6C shows a cross-section view of the air velocity profile shown in FIGS. 6A and 6B. The cross section is taken in a plane perpendicular to the horizontal air-output bottom surface of the plenum and intersects a center of the airflow enclosure of the plenum. As can be seen from FIG. 6C, an average air velocity of airflow in an outer region of airflow at a level of the patient treatment table (e.g., approximately 4-5 feet above the ground as indicated by dotted line 610) is appreciably higher than an average air velocity of airflow in an inner region of airflow produced by air emanating from the plurality of outlets (e.g., downward laminar airflow). An average velocity of airflow in a region (e.g., outer region 602 and inner region 604) may also be calculated over an entire volume of the region. The inner region 604 encompasses (e.g., surrounds) a patient treatment table 612 even when the patient is lying flat on the table. This outer region of airflow (that has higher average velocity than the average velocity of airflow in the inner region) defines a vertical curtain of air. In the cross-section of FIG. 6C, the vertical curtain of air (outer region) appears as the two red sub-regions of airflow (enclosed by dashed boxes) that are close to the perimeter of the air plenum airflow enclosure. The inner region (inside of the curtain and enclosed by the solid box) has a low average velocity of airflow.

In certain embodiments, a patient treatment table is approximately 3 feet, approximately 4 feet, or approximately 5 feet above the floor of the healthcare setting in which the patient treatment table and an air plenum are located. In certain embodiments, a patient treatment table is adjustable in height such that it may be adjusted within a range of 3-5 feet above the floor of a healthcare setting. In certain embodiments, an average velocity of airflow in an outer region of airflow is at least 1.5× higher (e.g., at least 2×, at least 3×, at least 4×, or at least 5× higher) than an average velocity of airflow in an inner region of airflow. In certain embodiments, airflow in an outer region of airflow and in an inner region of airflow is substantially downward laminar airflow produced by a plurality of air outlets in an air plenum. In certain embodiments, substantially downward laminar airflow produced by an air plenum is airflow having velocity vectors that have a larger downward component than horizontal component (e.g., wherein the magnitude of the downward component is at least 3 times larger or at least 8 times larger than the magnitude of the horizontal component).

In certain embodiments, average velocity of airflow in an outer region is at least 40 feet per minute (e.g., at least 45 feet per minute, at least 50 feet per minute, or at least 60 feet per minute) and average velocity of airflow in an inner region is no more than 30 feet per minute (e.g., no more than 25 feet per minute, no more than 20 feet per minute, or no more than 15 feet per minute) when measured at a height of no more than 6 feet (e.g., at a height of a patient treatment table). For example, referring to FIG. 6B, an outer region of airflow has an average velocity of airflow of approximately 35-40 feet per minute (fpm) while an inner region of airflow has an average velocity of airflow of approximately 7-10 fpm. In certain embodiments, an air plenum produces airflow (e.g., emanating from a plurality of air outlets) such that an average velocity of airflow in an outer region of airflow is at least 1.5× higher (e.g., at least 2×, at least 2.5×, at least 3×, at least 4×, or at least 5× higher) than an average velocity of airflow in an inner region of airflow.

Certain embodiments of the present disclosure were expressly described above. It is, however, expressly noted that the present disclosure is not limited to those embodiments, but rather the intention is that additions and modifications to what was expressly described herein are also included within the scope of the disclosure. Moreover, it is to be understood that the features of the various embodiments described herein were not mutually exclusive and can exist in various combinations and permutations, even if such combinations or permutations were not made express herein, without departing from the spirit and scope of the disclosure. In fact, variations, modifications, and other implementations of what was described herein will occur to those of ordinary skill in the art without departing from the spirit and the scope of the disclosure. As such, the disclosure is not to be defined only by the preceding illustrative description.

Having described certain implementations of methods and apparatus for air plenums and airflow through air plenums, it will now become apparent to one of skill in the art that other implementations incorporating the concepts of the disclosure may be used. Additionally, for example, certain embodiments of air plenums and methods of their use disclosed herein may be used outside of healthcare settings, for example in certain manufacturing or other settings (e.g., clean rooms) to maintain sterile areas in the settings (e.g., areas having a very low presence of contaminants). Therefore, for at least these reasons, the disclosure should not be limited to certain implementations, but rather should be limited only by the spirit and scope of the following claims.

1. A method of supplying a healthcare setting with airflow using an air plenum, the method comprising:
conducting air into the air plenum via one or more inlet air ducts, wherein the air plenum is installed in a ceiling of a healthcare setting and is structured such that the air conducted into the air plenum flows through a plurality of air outlets in the air plenum, wherein the plurality of air outlets are disposed within a bounded cross-sectional area and flowing the air through the plurality of air outlets produces airflow that is substantially downward and laminar,
wherein an outer region of the airflow has an average velocity of airflow that is at least 1.5× higher than an average velocity of airflow of an inner region of the airflow.

2. The method of claim 1, wherein the outer region and the inner region share a common center and the common center is a center of the air plenum.

3. The method of claim 1, wherein the air plenum is centered over a patient treatment table.

4. The method of claim 3, wherein the healthcare setting is an operating room and the patient treatment table is an operating table.

5. The method of claim 3, wherein the inner region encompasses the patient treatment table (e.g., such that the patient treatment table is contained within a vertical curtain of air effectively created by the air plenum and extending down from the perimeter of the bounded cross-sectional area).

6. The method of claim 1, wherein the average velocity of the outer region of the airflow at a height of a patient treatment table is at least 1.5× higher than the average velocity of the inner region of the airflow at the height of the patient treatment table.

7. The method of claim 1, wherein (i) the air plenum comprises a planar interior partitioning member and a plurality of interior air outlets that are disposed in the planar interior partitioning member and (ii) the planar interior partitioning member is spatially separated from and substantially parallel to the bounded cross-sectional area such that a pressure gradient is produced when conducting air into the air plenum.

8. The method of claim 1, wherein the bounded cross-sectional area is circular.

9. The method of claim 1, wherein the average velocity of airflow of the outer region of the airflow is an average of airflow velocity throughout a volume of the outer region and the average velocity of airflow in the inner region of the airflow is an average of airflow velocity throughout a volume of the inner region.

10. The method of claim 1, wherein the average velocity of airflow of the outer region of airflow is at least 40 feet per minute and the average velocity airflow of the inner region of airflow is no more than 30 feet per minute.

11. The method of claim 1, wherein the bounded cross-sectional area has a dimension of at least 4 feet.

12. The method of claim 1, wherein the bounded cross-sectional area is rectangular.

13. The method of claim 1, wherein the air plenum comprises one or more deflectors and each of the one or more inlet air ducts has a respective deflector of the one or more deflectors disposed nearby such that the air conducted through the one or more inlet air ducts is deflected by the one or more deflectors.

14. The method of claim 13, wherein the one or more deflectors deflect the air conducted into the air plenum via the one or more inlet air ducts such that the air has a velocity in a rotational direction that is substantially parallel to a horizontal air-output bottom surface of the air plenum, wherein the plurality of air outlets are disposed in the horizontal air-output bottom surface.

15. The method of claim 1, wherein the air plenum comprises a horizontal baffle and the method comprises:
conducting air into the plenum through one or more inlet air ducts;
flowing the air over the horizontal baffle; and
flowing the air through the plurality of air outlets after the air flows over the horizontal baffle.

16. The method of claim 1, wherein the air plenum is an integrated air and lighting plenum.

17. The method of claim 16, wherein one or more lights are each disposed in a respective housing in the integrated air and lighting plenum and the method comprises:
cooling the one or more lights by flowing air over the respective housing for each of the one or more lights, wherein the air flows over at least a portion of the respective housing prior to flowing through the plurality of air outlets.

18. The method of claim 17, wherein the respective housing of each of the one or more lights is disposed such that a circle centered at a center of the bounded cross-sectional area intersects a center of the respective housing.

19. The method of claim 1, wherein each of the plurality of air outlets has a circular cross section.

20. The method of claim 1, wherein conducting air into the air plenum via one or more inlet air ducts comprises:
conducting air through an electrostatic air filter, such that at least a portion of contaminants in the air are removed; and
subsequently, conducting air through a depth filter.

21. The method of claim 1, wherein the air plenum produces no more than 10 dB of noise (e.g., within the healthcare setting) while the air is conducted into the air plenum and flows through the plurality of air outlets.

22. The method of claim 1, wherein the air plenum produces no more than 2 dB of noise while the air is conducted into the air plenum and flows through the plurality of air outlets.

23-64. (canceled)
